(19) United States
(12) Patent Application Publication
(10) Pub. No.: US 2017/0209115 A1
(43) Pub. Date: Jul. 27, 2017

(54) METHOD AND SYSTEM OF SEPARATING AND LOCATING A PLURALITY OF ACOUSTIC SIGNAL SOURCES IN A HUMAN BODY

(71) Applicant: Quattro Folia Oy, Espoo (FI)

(72) Inventors: Pekka Juhani Lönnroth, Espoo (FI); Petteri Väisänen, Tampere (FI); Harri Okkonen, Espoo (FI); Ari Sinisalo, Helsinki (FI)

(73) Assignee: Quattro Folia Oy, Espoo (FI)

(21) Appl. No.: 15/412,022

(22) Filed: Jan. 22, 2017

Related U.S. Application Data
(60) Provisional application No. 62/286,476, filed on Jan. 25, 2016.

(51) Int. Cl. A61B 7/02; A61B 7/00; A61B 5/024; A61B 5/0408; A61B 5/00

(52) U.S. Cl. CPC A61B 7/02; A61B 5/0408; A61B 5/6804; A61B 5/024; A61B 5/7246; A61B 7/003; A61B 5/7275; A61B 2562/0219

(57) ABSTRACT

The invention provides a method and system of separating and locating a plurality of acoustic sources in a human body. The system includes a plurality of sensors, a processing unit and an output device. The method includes recording a plurality of signals by utilizing the plurality of sensors, wherein the plurality of sensors are arranged about one or more portions of the human body. A signal processing technique is applied for separating a plurality of sets of statistically independent sounds from the plurality of signals. Thereafter, a correlation technique is applied on the plurality of sets of statistically independent sounds to obtain a plurality of sets of reference signal dependent sounds. Finally, an advance acoustic location technique is applied on each of the plurality of sets of reference signal dependent sounds to locate the plurality of acoustic signal sources.

### FIELD OF THE INVENTION

The invention generally relates to recording and analysis of human body sounds. More specifically, the invention relates to a method and system of separating and locating naturally occurring acoustic sources in a human body, for diagnostic applications and further estimating the risk associated with future diseases.

### BACKGROUND OF THE INVENTION

Typically, there are many sources of sounds in a human body that are related to the function of organs such as, but not limited to, heart, lungs, blood circulation, and ingestion system. Some sounds are normal and some of the sounds are associated with a sickness, dysfunction or abnormal situations. In addition, the sound can change during different diseases and that can help in the diagnosis or follow-up of the diseases. Typically, auscultation with a stethoscope is a normal practice of physicians to listen to lungs and hearts for evaluating condition of the lungs and hearts. Based on the auscultation, a physician can find information to support diagnosis of diseases. The physician can recognize and differentiate different sounds by moving the stethoscope and finding changes in sound levels. Based on the experience of the physician, the physician judges if the sounds or change in the sounds levels are normal or related to a disease or a dysfunction. The auscultation with a stethoscope is a supporting tool for a physician and usually recordings of auscultation are not made. Thus, information about the sounds is not collected and in turn, the information is not available for later reference. In addition, a patient cannot go for a second opinion about that particular situation from another physician.

Thus, there is a need to develop an improved system and method for separating and locating acoustic sources in a human body.

### BRIEF DESCRIPTION OF THE FIGURES

The accompanying figures where like reference names refer to identical or functionally similar elements throughout the separate views and which together with the detailed description below are incorporated in and form part of the specification, serve to further illustrate various embodiments and to explain various principles and advantages all in accordance with the invention.

FIG. 1 illustrates a block diagram of a system for separating and locating a plurality of acoustic sources in a human body in accordance with various embodiments of the invention.

FIG. 2 illustrates a flow diagram of a method for separating and locating a plurality of acoustic sources in a human body in accordance with various embodiments of the invention.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the invention.

### DETAILED DESCRIPTION OF THE INVENTION

Before describing in detail embodiments that are in accordance with the invention, it should be observed that the embodiments are primarily for a method and system of separating and locating a plurality of acoustic sources in a human body. Accordingly, components of the partitioned table have been represented where appropriate by conventional symbols in the drawings, showing only those specific details that are pertinent to understanding the embodiments of the invention so as not to obscure the disclosure with details that will be readily apparent to those of ordinary skill in the art having the benefit of the description herein.

In this document, the terms “comprises,” “comprising,” or any other variation thereof, are intended to cover a non-exclusive inclusion, such that a process, method, article or apparatus that comprises a list of objects does not include only those objects but may include other objects not expressly listed or inherent to such process, method, article, or apparatus. An object proceeded by “comprises . . . a” does not, without more constraints, preclude the existence of additional identical objects in the process, method, article, or apparatus that comprises the object.

Various embodiments of the invention provide a method and system of separating and locating a plurality of acoustic sources in a human body. The system includes a plurality of sensors, a processing unit and an output device. The method includes recording a plurality of signals by utilizing the plurality of sensors, wherein the plurality of sensors are disposed in a plurality of portions of the human body. The plurality of sensors may be attached to the body surface, located inside of the body, e.g., swallowed, or be a remote sensing system, e.g., based on Doppler radar. Some or all of the sensors may be also active acoustical transmitters to locate the sensor, e.g., in the stomach or gut. Transmitting sensors may be used to calibrate the system, too. A signal processing technique is applied for separating a plurality of sets of statistically independent sounds from the plurality of signals. Thereafter, a correlation technique is applied on the plurality of sets of statistically independent sounds to obtain a plurality of sets of reference signal dependent sounds. Finally, an acoustic source location is applied to the separated signals. The location can be based on, e.g., time-delay estimation (TDE) methods such as the generalized cross-correlation (GCC) function, which estimates location based on the time delays of the arriving signals at the receivers, or some direct methods such as steered beam-forming or more preferably some modern technique such as Sequential Monte-Carlo (SMC) method, also known as Particle Filtering (PF) which is applied on each of the plurality of sets of reference signal dependent sounds to locate the plurality of acoustic signal sources.

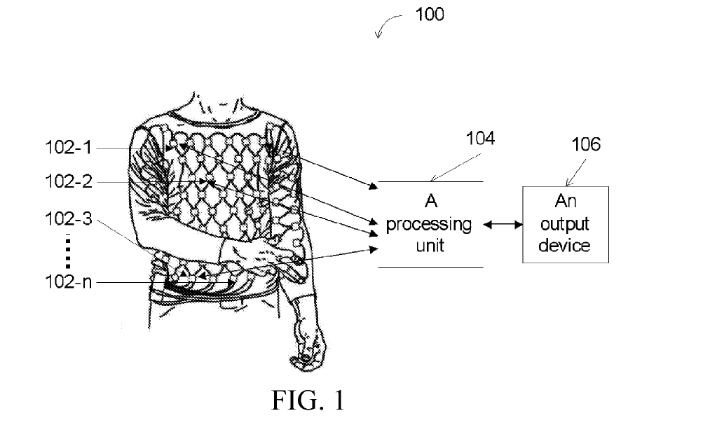

Referring to FIG. 1, a system 100 for separating and locating a plurality of acoustic sources in a human body is illustrated in accordance with various embodiments of the invention. System 100 includes a plurality of sensors 102 (1-n), a processing unit 104 and an output device 106.

Plurality of sensors 102 (1-n) are disposed in a plurality of portions of the human body. Each of the plurality of sensors 102 (1-n) is one of a microphone, a stretch sensor, an accelerometer and two or more of ECG electrodes with a ground electrode and leads. In an embodiment, plurality of sensors 102 (1-n) are each disposed as a mesh about one or more portions of the human body. Plurality of sensors 102 (1-n) are configured to record a plurality of signals.

Processing unit 104 is coupled to plurality of sensors 102 (1-n). Processing unit 104 is configured to perform one or more of, but not limited to, applying a signal processing technique for separating a plurality of sets of statistically independent sounds from the plurality of signals, applying a correlation technique on the plurality of sets of statistically independent sounds to obtain a plurality of sets of reference signal dependent sounds, and applying an advance acoustic location technique on each of the plurality of sets of reference signal dependent sounds to locate the plurality of acoustic signal sources.

Output device 106 is coupled to processing unit 104. Output device 106 is configured to display the location of each of the plurality of acoustic signal sources.

In another embodiment, processing unit 104 is further configured to compare one or more external sound sources with the plurality of acoustic signals located in the human body. The one or more external sources are attached to a portion of the human body, wherein the external sound sources may include sinusoidal signals with known frequency or wideband noise. The external sound sources may be recorded simultaneously with the naturally occurring acoustic sources as located in the human body. Furthermore, the recorded external sound sources are compared with the naturally occurring acoustic sources, for calculating the transfer functions between different locations and portions in the human body along with change in transfer functions over time. Based on the calculations, transfer function trend may be calculated to be employed in diagnostic applications. The transfer function trend may also be employed in estimating risk associated with future diseases. In one example, the transfer function trend may be employed in the follow-up of development of pneumothorax when an abnormal collection of air exists in the pleural space that causes an uncoupling of the lung from the chest wall.

The external sound sources may also be recorded separately from the acoustic sources located in the human body. The recordings of the separated external sound sources and the located sources of the naturally acoustic sounds are stored in a cloud based storage device, which may be further employed in statistical analysis, correlations or generating more advanced mathematical models. Mathematical models which may include but is not limited to Monte Carlo Tree Search, Neural Network Optimization or any other Artificial Intelligence is used to establish correlations between different life styles over population and over time to find potential development paths and links between found sounds and future diseases, e.g., some breathing related sounds may indicate very early stage asthma or COPD (Chronic Obstructive Pulmonary Disease).

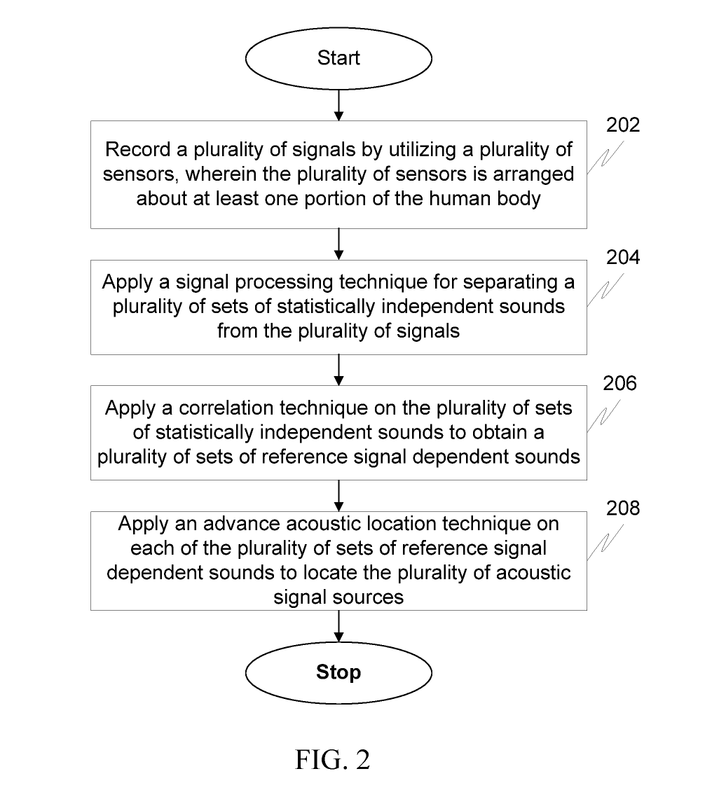

Referring to FIG. 2, a method for separating and locating a plurality of acoustic sources in a human body is illustrated in accordance with various embodiments of the invention.

At step 202, a plurality of signals are recorded utilizing a plurality of sensors. The plurality of sensors are disposed in a plurality of portions of the human body. Each of the plurality of sensors is one of a microphone, a stretch sensor, an accelerometer and two or more of ECG electrodes with a ground electrode and leads. For example, small microphones and acceleration sensors are attached to the skin on different location of the human. The small microphones and acceleration sensors are attached to mainly the thorax portion and over main arteries on wrists and ankles. The integrated acceleration sensors with each microphone are used to measure local movements of the human body. For example, the local movement measurement can be related to each heartbeat to measure the cardiac output. The microphones in wrists and ankles are located over the arteries to record heartbeats using these locations.

In one embodiment, the plurality of sensors are attached to the human body using a special pad with two sided glue and acoustic gel to improve the coupling between human body and the sensors. A stretching mesh network of the plurality of stretch sensors can also be used. An additional benefit of the stretching mesh network is that relative distances of the stretch sensors can be same and the stretch sensors also provide information about the human body measures and a reference breath signal.

At step 204, a signal processing technique is applied for separating a plurality of sets of statistically independent sounds from the plurality of signals. The signal processing technique may include one of an Independent Component Analysis (ICA), a Fast Independent Component Analysis (FICA) or any other advanced technique. Plurality of signals include one or more of a plurality of acoustic signals, a plurality of stretch sensor signals, a plurality of accelerometer signals and a plurality of ECG recordings.

Thereafter, at step 206, a correlation technique is applied to the plurality of sets of statistically independent sounds to obtain a plurality of sets of reference signal dependent sounds. The applying of the correlation technique includes utilizing one or more of a reference ECG signal and a reference breath signal. The reference may be from an external source like from a metronome, wave or noise generator.

Finally, at step 208, an advance acoustic location technique is applied on each of the plurality of sets of reference signal dependent sounds to locate the plurality of acoustic signal sources. Each of the plurality of sets of reference signal dependent sounds is one of a set of ECG correlated sounds and a set of non-ECG correlated sounds. The applying of the correlation technique includes categorizing each of the plurality of sets of reference signal dependent sounds as one of the set of ECG correlated sounds and the set of non-ECG correlated sounds. The set of non-ECG correlated sounds is one of a set of breath correlated sounds and a set of non-ECG non-breath correlated sounds. The applying of the correlation technique further includes categorizing each set of non-ECG correlated sounds as one of the set of breath correlated sounds and the set of non-ECG non-breath correlated sounds. The applying of the advance acoustic location technique includes utilizing a plurality of stretch signals for separating and locating one or more of the sources of the set of ECG correlated sounds, the source of the set of breath correlated sounds, and the source of non-ECG non-breath correlated sounds. The external reference may be utilized in detection of variations in body sound patterns.

In another embodiment, the method further includes attaching one or more external sound sources attached to a plurality of portions of the human body, wherein the external sound sources may include sinusoidal signals with known frequency or wideband noise. The external sound sources may be recorded simultaneously with the naturally occurring acoustic sources as located in the human body. Furthermore, the recorded external sound sources are compared with the naturally occurring acoustic sources, for calculating the transfer functions between different locations and portions in the human body along with change in transfer functions over time. Based on the calculations, transfer function trend may be calculated to be employed in diagnostic applications. The transfer function trend may also be employed in estimating risk associated with future diseases.

Various embodiments of the invention provide support in determining different normal and abnormal situations associated with a human body by recording and analyzing different sounds in the human body. For example, heartbeat recordings at the wrists and ankles of a human body can be used to analyze conditions of arteries. The time of the arrival and the power spectrum of the heartbeat recordings indicate the stenosis and hardness of arteria. Stenosis of a vascular type is often associated with unusual blood sounds resulting from a turbulent flow over a narrowed blood vessel. Thus, recordings can be used to locate stenosis. In another example, a diabetic person can utilize the invention to define the critical blood glucose levels when the blood vessels start to react to high blood glucose levels. In addition, Heart Rate variability (HRV) can be linked to other changes such as changes in blood vessel properties to determine different normal and abnormal situations.

Another application of the invention is follow-up of development of infants and small babies. Recordings can be used to follow swallowing during breastfeeding so that mother is able to follow and learn her child's behavior and development. When the recordings are stored in a cloud based storage device, the parents can check and compare different statistics against a larger reference group and look for help if clear differences become visible.

Those skilled in the art will realize that the above-recognized advantages and other advantages described herein are merely exemplary and are not meant to be a complete rendering of all of the advantages of the various embodiments of the invention.

In the foregoing specification, specific embodiments of the invention have been described. However, one of ordinary skill in the art appreciates that various modifications and changes can be made without departing from the scope of the invention. Accordingly, the specification and figures are to be regarded in an illustrative rather than a restrictive sense, and all such modifications are intended to be included within the scope of the invention. The benefits, advantages, solutions to problems, and any element(s) that may cause any benefit, advantage, or solution to occur or become more pronounced are not to be construed as a critical, or required.

What is claimed is:

1. A method for locating a plurality of acoustic signal sources in a human body, the method comprising:
recording a plurality of signals by utilizing a plurality of sensors, wherein the plurality of sensors are disposed in a plurality of portions of the human body;
applying a signal processing technique for identifying a plurality of sets of statistically independent sounds from the plurality of signals;
applying a correlation technique on the plurality of sets of statistically independent sounds to obtain a plurality of sets of reference signal dependent sounds; and
applying a beam-forming technique on each of the plurality of sets of reference signal dependent sounds to locate the plurality of acoustic signal sources.

2. The method of claim 1, wherein the plurality of signals comprises at least one of a plurality of acoustic signals, a plurality of stretch sensor signals, a plurality of accelerometer signals and a plurality of ECG signals.

3. The method of claim 1, wherein each of the plurality of sensors is one of a microphone, a stretch sensor, an accelerometer and an ECG probe.

4. The method of claim 1, wherein the signal processing technique is one of Independent Component Analysis (ICA) and Fast Independent Component. Analysis (FICA)

5. The method of claim 1, wherein applying the correlation technique comprises utilizing at least one of a reference ECG signal, a reference accelerometer signal, a reference stretch signal and an external reference signal.

6. The method of claim 1, wherein each of the plurality of sets of reference signal dependent sounds is one of a set of ECG correlated sounds and a set of non-ECG correlated sounds.

7. The method of claim 6, wherein applying the correlation technique comprises categorizing each of the plurality of sets of reference signal dependent sounds as one of the set of ECG correlated sounds and the set of non-ECG correlated sounds.

8. The method of claim 7, wherein the set of non-ECG correlated sounds is one of a set of breath correlated sounds and a set of non-ECG non-breath correlated sounds.

9. The method of claim 8, wherein applying the correlation technique further comprises categorizing each set of non-ECG correlated sounds as one of the set of breath correlated sounds and the set of non-ECG non-breath correlated sounds.

10. The method of claim 9, wherein applying the beam-forming technique comprises utilizing a plurality of stretch signals for locating at least one of the source of the set of ECG correlated sounds, the source of the set of breath correlated sounds, and the source of non-ECG non-breath correlated sounds.

11. The method of claim 1 further comprising attaching at least one external sound source to a portion of the human body, wherein the at least one external sound source is simultaneously recorded with the plurality of acoustic signals sources in the human body.

12. The method of claim 11, wherein the at least one external sound source is recorded separately from the plurality of acoustic signals sources in the human body.

13. The method of claim 11 further comprising comparing the at least one external sound source with the plurality of acoustic signals sources in the human body for diagnostic applications.

14. The method of claim 13, wherein the at least one external sound source is compared with the plurality of acoustic signals sources in the human body for estimating a risk associated with future diseases.

15. The method of claim 13, wherein the at least one external sound sources are recorded and stored in a cloud based storage device.

16. A system for locating a plurality of acoustic signal sources in a human body, the system comprising:
a plurality of sensors disposed in a plurality of portions of the human body, wherein the plurality of sensors are configured to record a plurality of signals;
a processing unit coupled to the plurality of sensors, wherein the processing unit is configured to perform at least one of:
applying a signal processing technique for identifying a plurality of sets of statistically independent sounds from the plurality of signals;
applying a correlation technique on the plurality of sets of statistically independent sounds to obtain a plurality of sets of reference signal dependent sounds; and
applying a beam-forming technique on each of the plurality of sets of reference signal dependent sounds to locate the plurality of acoustic signal sources; and
an output device coupled to the processing unit, wherein the output device is configured to display the location of each of the plurality of acoustic signal sources, wherein the plurality of located acoustic signal sources are stored in a cloud based storage device.

17. The system of claim 16 further comprising at least one external sound source attached to a portion of the human body, wherein the at least one external sound source is simultaneously recorded with the plurality of acoustic signals sources in the human body.

18. The system of claim 18, wherein the at least one external sound source is recorded separately from the plurality of acoustic signals sources in the human body.

19. The system of claim 18, wherein the processing unit is configured to compare the at least one external sound source with the plurality of acoustic signals sources in the human body for diagnostic applications.

20. The system of claim 18, wherein the processing unit is configured to compare the at least one external sound source with the plurality of acoustic signals sources in the human body for estimating a risk associated with future diseases.
